(19) United States
(12) Patent Application Publication
(10) Pub. No.: US 2025/0363407 A1
(43) Pub. Date: Nov. 27, 2025

(54) METHOD FOR SOIL AND ROCK CLASSIFICATION BASED ON DUAL-PARAMETER CLUSTERING ANALYSIS

(71) Applicants: Zijian Zhang, Katy, TX (US); Ya Li, Katy, TX (US); Zhengbo Jiang, Shenzhen (CN); Cun Hu, Shenzhen (CN); Luwei Sun, Shenzhen (CN); Xishun Pang, Shenzhen (CN); Fanli Xu, Katy, TX (US)

(72) Inventors: Zijian Zhang, Katy, TX (US); Ya Li, Katy, TX (US); Zhengbo Jiang, Shenzhen (CN); Cun Hu, Shenzhen (CN); Luwei Sun, Shenzhen (CN); Xishun Pang, Shenzhen (CN); Fanli Xu, Katy, TX (US)

(73) Assignees: Res. Inst. Of Tsinghua Univ. In Shenzhen, Shenzhen (CN); Prime Ocean Technology (Shenzhen) Inc., Katy, TX (US)

(21) Appl. No.: 18/671,852

(22) Filed: May 22, 2024

(51) Int. Cl. G06N 20/00; G01N 33/24

(52) U.S. Cl. CPC G06N 20/00; G01N 33/24

(57) ABSTRACT

A method for soil and rock classification using dual-parameter cluster analysis is provided. The method improves current classification techniques by incorporating both mechanical and physical parameters. The process involves four key steps by applying a machine learning processing: (a) data acquisition, preprocessing, and feature extraction to obtain dual-parameter data, which are then divided into training and testing sets; (b) construction of a dual-parameter cluster model using cluster analysis algorithms and performing clustering on the training dataset; (c) formulation of classification standards based on clustering results, and verification using the testing dataset; and (d) application of the model to classify new soil and rock data once accuracy criteria are met. This method obviously enhances the accuracy and efficiency of soil and rock classification and is suitable for various geological applications.

### FIELD OF THE INVENTION

The invention relates to the field of soil and rock classification technology, particularly to a method for soil and rock classification based on dual-parameter cluster analysis.

### BACKGROUND OF THE INVENTION

The classification and description of soil are important in site investigation. However, globally, there is a lack of uniform standards for soil and rock classification. Different countries and regions have different methods. For example, clay, a common soil encountered in offshore site investigations, is classified differently under various national standards. The American Society for Testing and Materials (ASTM) standards D2487 and D2488 are widely used but are somewhat subjective and demand high experiences. Field engineers must classify clay based on tactility into categories such as very soft, soft, firm, stiff, very stiff, and hard. In contrast, the petroleum and natural gas industry standards in Country A use a mechanical parameter called undrained shear strength to classify clay into several categories from very soft to hard. Moreover, the housing and urban-rural development standard in Country A classifies clay based on a physical parameter like the liquidity index, dividing it into categories such as hard, hard plastic, plastic, soft plastic, and fluid. These varying methods and principles can lead to significant discrepancies and confusion in soil classification within the same survey area.

### SUMMARY OF THE INVENTION

The primary objective of this invention is to provide a method for soil and rock classification based on dual-parameter cluster analysis, aimed at addressing the inefficiencies and inaccuracies in current classification methods which do not adequately consider both mechanical and physical parameters.

To achieve the above objectives, the invention proposes a method for soil and rock classification based on dual-parameter clustering analysis, comprising the steps of:
a. Data Acquisition: Collecting soil data, preprocessing this data, and extracting feature parameters to obtain dual-parameter data which is then divided into training and testing datasets.
b. Model Construction: Building a dual-parameter cluster model by using cluster analysis algorithm, and then performing cluster analysis on the training dataset to obtain clustering results.
c. Standard Formulation: Based on the clustering results and geological analysis, formulating classification standards, testing these standards with the test dataset, and adjusting the classification standards to obtain accurate results.
d. Result Application: When the classification accuracy meets predefined criteria, other soil and rock data are input into the dual-parameter cluster model to achieve the classification results.

The invention uses cluster analysis algorithms to analyze collected soil and rock data, enabling rapid and effective identification of relationships between soil and rock data, thereby accurately and automatically classifying different types of soil and rock.

### BRIEF DESCRIPTION OF THE DRAWINGS

For a detailed description of the preferred embodiments of the invention, reference will now be made to the accompanying drawings in which:

FIG. 1 is a structural diagram of the soil and rock classification method according to an embodiment of the present invention.

FIG. 2 is a Schematic of clustering components used for soil and rock classification according to an embodiment of the present invention.

FIG. 3 is a structural diagram of data preprocessed according to an embodiment of the present invention.

FIG. 4 is a structural diagram of data cleaning and transformation according to an embodiment of the present invention.

FIG. 5 is a structural diagram of the model construction according to an embodiment of the present invention.

FIG. 6 is a flow chart depicting a process of K-means clustering algorithm.

FIG. 7 is a structural diagram of the first portion in the K-means clustering algorithm according to an embodiment of the present invention.

FIG. 8 is a structural diagram of the second portion in the K-means clustering algorithm according to an embodiment of the present invention.

FIG. 9: is a structural diagram of classification verification module according to an embodiment of the present invention.

FIG. 10 shows the undrained shear strength data and the corresponding classification based on a single mechanical parameter for each station.

FIG. 11 shows showing the undrained shear strength and submerged unit weight dual-parameter plot according to an embodiment of the present invention.

FIG. 12 is the classification result of the preferred embodiment of the soil and rock classification method based on dual-parameter cluster analysis of this invention.

FIG. 13 is the classification verification result of the preferred embodiment of the soil and rock classification method based on dual-parameter cluster analysis of this invention.

### DETAILED DESCRIPTION OF EMBODIMENTS

Existing methods for geological description of soil and rock in engineering projects have limitations, especially evident in offshore wind farm surveys involving numerous core samples and Cone Penetration Test (CPT) data. In site investigations, geological descriptions are primarily based on observations and manual tactility by site engineers, which are highly subjective, such as the thumb penetration method depicted in ASTM standards. Although pocket penetrometers are occasionally used to indicate the state of the clay on-site, there is a general lack of standardized and quantitative methods. During the later stages of laboratory analysis, even though experimental results can revise on-site geological descriptions, the industry standards and codes are relatively simplistic and do not fully consider the impact of both mechanical and physical parameters, thus leading to low efficiency and inaccuracies in soil analysis.

Additionally, some researchers have tried to develop soil and rock classification diagrams based on the Cone Penetration Test (CPT), which, although unable to measure the soil and rock properties directly, reflects their strength and stiffness parameters through sensor data from the penetration probe. The advantage of CPT is its ability to measure continuously at a single probe location, avoiding the cumbersome steps of laboratory sample preparation. However, this method remains overly simplistic in classification, primarily based on mechanical parameters and ignoring the impact of various other geological parameters. In reality, the load response of soil and rock is influenced by multiple factors such as depositional processes, stress history, and chemical and biological processes, which can limit the classification effectiveness of the CPT method in complex scenarios. If the existing CPT soil and rock classification diagrams are not further refined, they are not suitable for offshore soil and rock.

Shortly, traditional methods for classifying and describing soil and rock face many issues and need improvement. Therefore, this invention proposes a soil and rock classification method based on dual-parameter cluster analysis, aiming to overcome the limitations of existing methods and enhance the accuracy, comprehensiveness, and consistency of soil and rock classification and description.

This invention uses artificial intelligence technology and employs clustering algorithms to analyze large volumes of geotechnical/geological data. Specifically, multiple geotechnical parameter data collected are input into the clustering algorithm. Through the algorithm's learning and training processes, the method achieves automatic classification of different types of soil and rock. Compared to traditional methods, the artificial intelligence method based on dual-parameter cluster analysis can capture the relationships between geotechnical/geological data more effectively and quickly, thereby achieving more accurate divisions of different soil and rock types. Further, intelligent classification is compared and revised against original geological descriptions to correct any errors. Hereinafter, an embodiment of the present invention is described in detail with reference to the drawings.

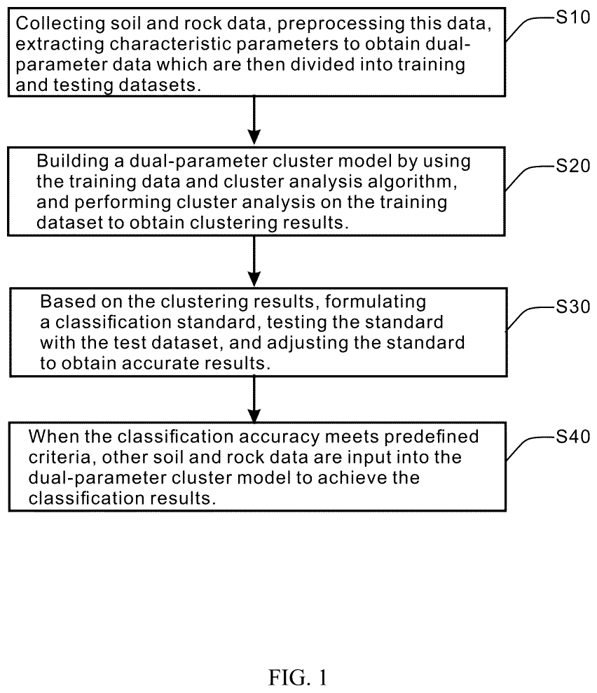

FIG. 1 is a structural diagram of dual-parameter cluster analysis according to an embodiment of the present invention.

As shown in FIG. 1, a dual-parameter cluster analysis includes four steps, which are preprocessing data and extracting feature parameters (S10), building a dual-parameter cluster model (S20), formulating and testing classification standards (S30), and applying the standards to preprocessed data (S40).

Step S10 is to acquire soil data, preprocess, and extract feature parameters to obtain dual-parameter data, which is then divided into a training set and a testing set.

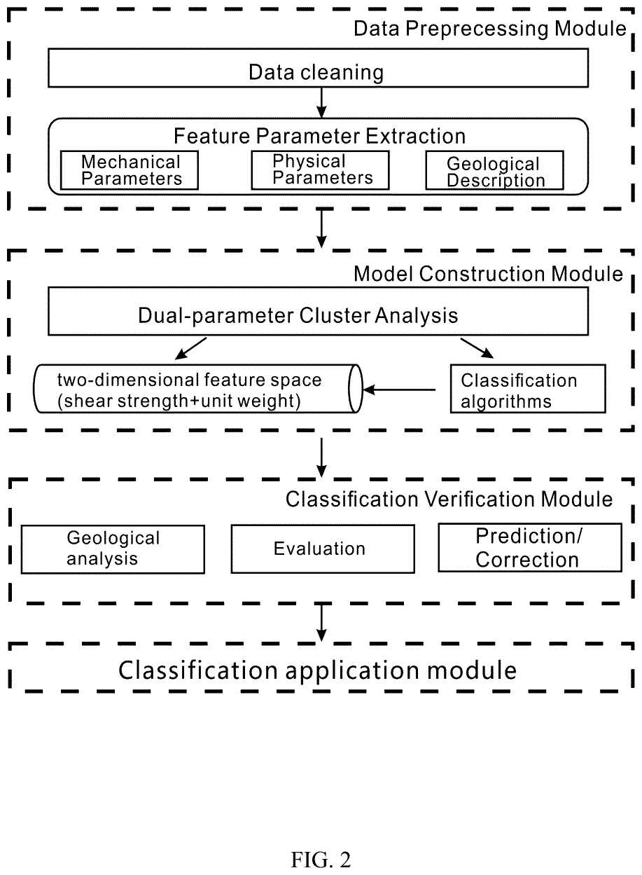

Step S10 corresponds to the Data Preprocessing Module shown in FIG. 2. By field survey and laboratory testing, this invention has collected data of soil and rock from site investigation. The data include various geotechnical parameters and geological description of soil samples. All data fall under geological/geotechnical parameters, which include three main categories: geological description, mechanical parameters, and physical parameters, interrelated to each other. one aim of this invention is to determine or correct some geological descriptions using mechanical and physical parameters.

Data Preprocessing consists of cleaning and feature parameter extraction. Considering the possible presence of noise and missing values in the collected data, data preprocessing is taken. This segment includes data cleaning and filling missing values, ensuring data accuracy and completeness. Further, from the processed data, two types of feature parameters are extracted: mechanical and physical parameters, such as undrained shear strength and submerged unit weight, which play a key role in subsequent analysis.

Step S20 is to build a dual-parameter cluster model using the training dataset and clustering analysis algorithm, then, perform cluster analysis on the training dataset to obtain clustering results. It corresponds to the model construction module shown in FIG. 2.

This invention introduces artificial intelligence clustering analysis technology, feeding the preprocessed data into a dual-parameter cluster analysis system to construct an innovative dual-parameter cluster model. By establishing a dual-parameter database and a two-dimensional feature space, the Model Construction Module classifies geotechnical samples comprehensively in the undrained shear strength and submerged unit weight. In this module, various classification algorithms, such as support vector machines, neural networks, KNN, hierarchical clustering, etc., can be used to construct this cluster model. This example uses the K-means algorithm, but the invention is not limited to this algorithm.

Step S30 is to formulate classification standards based on the clustering results, geologically analyze clustering results to define classification standards, test these standards with the testing dataset to validate results, and adjust the classification standards based on these results. It corresponds to the classification verification module shown in FIG. 2.

In the module, data have been divided into training and testing datasets, allowing for the assessment of the dual-parameter cluster model's classification accuracy and reliability using the testing dataset. The original classification standard is subject to updates. The module is composed of geological analysis, validation, and correction/prediction. After applied geological analysis and test data validation, predicted data are evaluated and final classification standards are established.

Step S40 is the final step, corresponding to the classification application module shown in FIG. 2. Once the accuracy meets predefined criteria, soil and rock data are input into the dual-parameter cluster model to generate classification results.

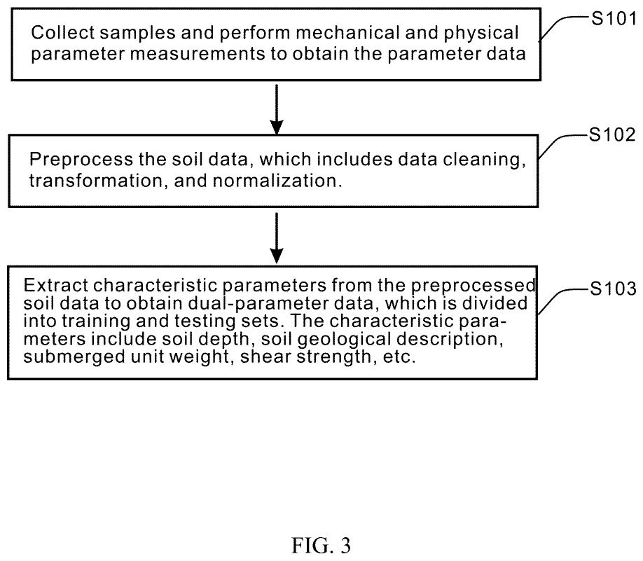

Referring to FIG. 3, in an embodiment, the data preprocessing module in S10 further includes:
S101: Specifically, collect soil and rock samples and perform mechanical and physical parameter measurements to obtain the parameter data.
S102: Preprocess the soil data, which includes data cleaning, data transformation, and data normalization.
S103: Extract feature parameters from the preprocessed soil and rock data to obtain dual feature parameter data, which is then divided into training and testing sets. The feature parameters include soil depth, soil geological description, submerged unit weight, and design shear strength.

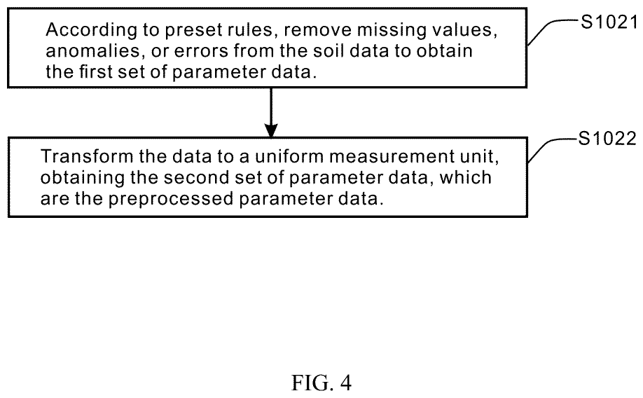

Referring to FIG. 4, data cleaning and transformation in S102 is divided into two segments:
S1021: According to preset rules, remove missing values, anomalies, or errors from the soil data to obtain the first set of parameter data.
S1022: Transform the data to a uniform measurement unit, obtaining the second set of parameter data, which are the preprocessed parameter data.

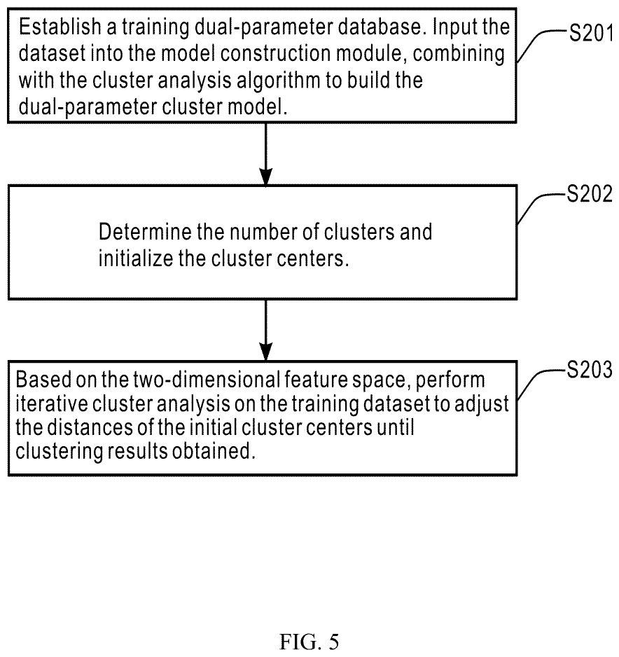

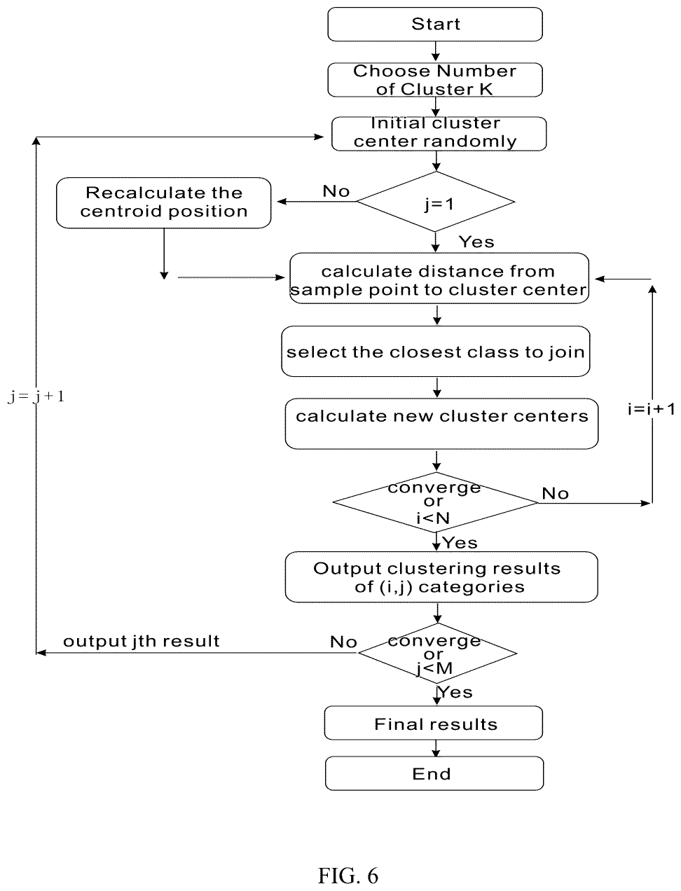

Referring to FIG. 5, in an embodiment, the model construction module in S20 further includes:
S201: Establish a training dual-parameter database. Input the dataset into the model construction module, combining with the cluster analysis algorithm to build the dual-parameter cluster model.
S202: Determine the number of clusters based on the cluster model and initialize the cluster centers.
S203: Based on the two-dimensional feature space, perform iterative cluster analysis on the training dataset to adjust the distances of the initial cluster centers until clustering results obtained (FIG. 6).

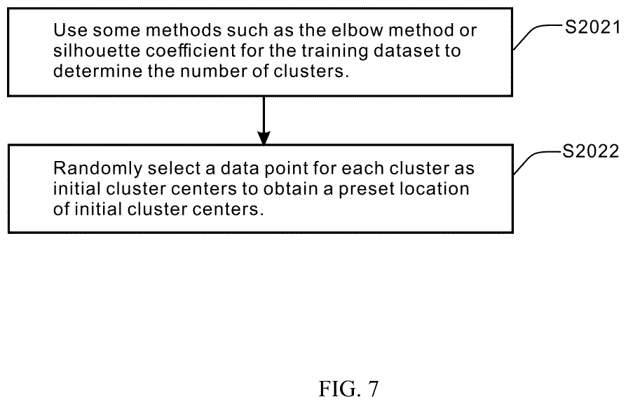

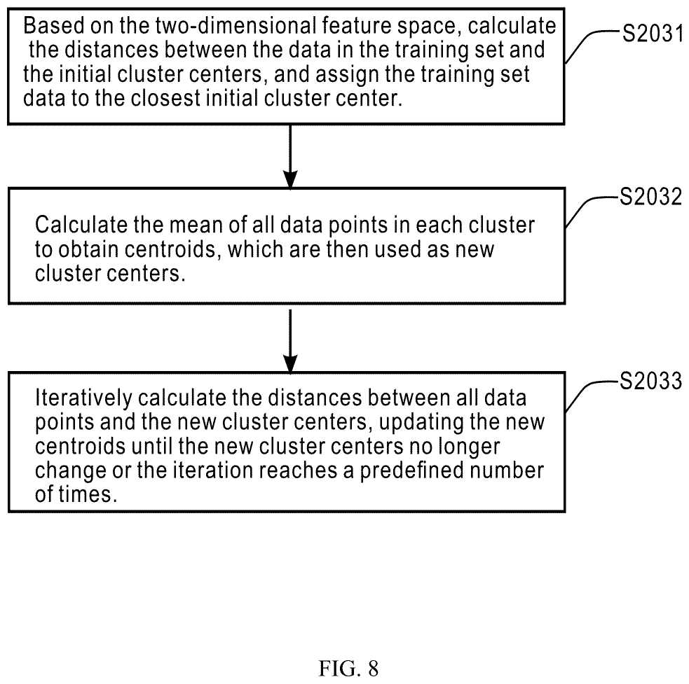

Referring to FIG. 7, S202 consists of two segments:
S2021: Based on the dual-parameter cluster model, use some algorithm methods such as the elbow method or silhouette coefficient for the training set to determine the number of clusters.
S2022: Based on the cluster analysis algorithm, randomly select a data point for each cluster as initial cluster centers to obtain a preset location of initial cluster centers. Referring to FIG. 8, S203 in this embodiment further includes:
S2031: Based on the two-dimensional feature space in the dual-parameter cluster model, calculate the distances between the data in the training set and the initial cluster centers, and assign the training set data to the closest initial cluster center, obtaining initial clustering results.
S2032: Calculate the mean of all data points in each cluster to obtain centroids, which are then used as new cluster centers.
S2033: Iteratively calculate the distances between all data points in the training set and the new cluster centers, updating the new centroids based on these distances until the new cluster centers no longer change or the iteration reaches a predefined number of times, thus obtaining the clustering results.

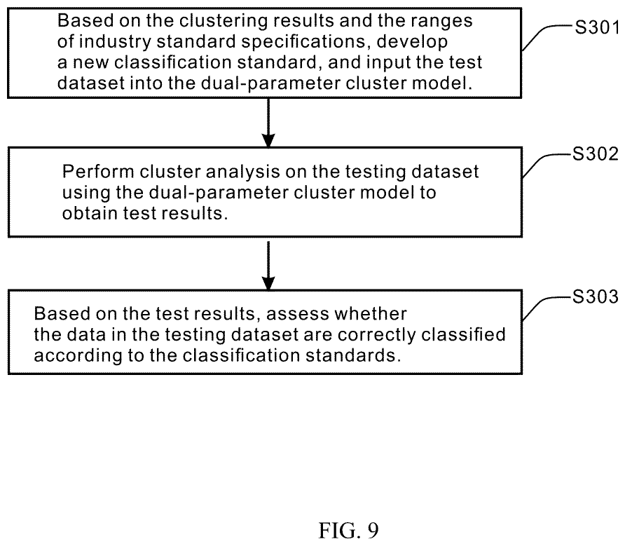

Referring to FIG. 9, the classification verification module in S30 further includes:
S301: Specifically, based on the clustering results and the ranges of industry standard specifications, develop a new classification standard, and input the testing dataset into the dual-parameter cluster model.
S302: Perform cluster analysis on the testing dataset using the dual-parameter cluster model to obtain test results.
S303: Based on the test results, assess whether the data are correctly classified according to the classification standards and whether the classification is geologically logical. If these conditions are met, the classification standard is deemed to have fulfilled the preset requirements. If not, return to S1021 to reprocess the data.

Through the steps mentioned above, the soil and rock classification system and method of this invention can more accurately delineate different geological/geotechnical categories, providing a new, rapid, and reliable method for geological/geotechnical classification for site investigation, such as offshore engineering. This method, by employing dual-parameter cluster analysis, comprehensively considers the combined effects of multiple geological parameters, avoiding the overly simplified single-parameter classification issues present in traditional methods, thereby enhancing the accuracy and practicality of the classification.

### EXAMPLE APPLICATION

The invention is illustrated by an example of site investigation in a specific marine area. This example describes in detail the implementation scheme of the soil classification system and method based on dual-parameter cluster analysis. It demonstrates the efficiency and accuracy of the method.

The first step is S10, which is to acquire soil data, preprocess, and extract feature parameters for a dual-parameter domain. In this instance, a service company has completed a site investigation for one offshore wind farm in Country A, and collected borehole and CPT data at 11 locations. For the study of clay, manual and electric vane tests were used onboard. After the tests, a hydraulic pusher was used to extract the soil samples from the sampling tubes, which were then visually identified, classified, and described by site engineers. Representative soil samples were placed in airtight containers and subsequently sent to an onshore laboratory for further testing. Laboratory tests included bulk density tests, moisture content tests, relative density tests, liquid and plastic limit tests, granulometry tests, electric vane tests, unconsolidated-undrained (UU) triaxial compression tests, consolidated-drained (CD) multistage triaxial compression tests, and consolidated-undrained (CU) multistage triaxial compression tests. Therefore, the mechanical parameter data collected by this invention include manual vane tests, electric vane tests, UU triaxial compression tests, CD multistage triaxial compression tests, and CU multistage triaxial compression tests; physical parameter data include bulk density tests, moisture content tests, relative density tests, liquid and plastic limit tests, and granulometry tests obtained through these experiments.

To ensure the accuracy and reliability of the data, this invention performed preprocessing on the collected geotechnical data. First, data cleaning was conducted, particularly for the clay data, to eliminate any potential missing values, anomalies, or incorrect data. When anomalies were detected, appropriate adjustments were made according to predetermined rules (based on the general distribution ranges of mechanical and rock parameters). For example, data indicating an underwater unit weight of 10.1 KN/m³ was excluded. After further verification, the excluded section of sample was confirmed to be composed of silt.

Secondly, data transformation and normalization were another crucial step in preprocessing to ensure data from various sources and formats could be uniformly compared and analyzed. Bulk density is expressed in kilograms per cubic meter in International System of Units (SI). For example, the bulk density of water is roughly 1000 kg/m³. In the site investigation, it is customary to multiply the density by the gravitational acceleration (g), converting it to a unit of kN/m³, e.g., the bulk density of water being approximately 9.8 kN/m³. Shear strength is commonly measured in kilopascals (kPa) and kilopounds per square foot (ksf), where 1 kPa is approximately equal to 0.020885434273039 ksf. This invention converted the units of bulk density from the International System's kg/m³ to the more commonly used kN/m³ for site investigation, and normalizing shear strength to kPa. This step ensured uniformity and comparability of the data.

The results from the field and onshore laboratory tests were summarized in tables, which include the soil description and soil parameters within the drilling depths at each borehole and CPT test site. For each site, a single design parameters table was established, such as Table 1.

TABLE 1
Soil classification, description, and designed parameters at one borehole

| | | Penetration(m) | | Thickness | Unit | Shear | Relative |
|---|---|---|---|---|---|---|---|
| Stratum | Description | From | To | (m) | weight | Strength | Density |
| 1 | Very soft to soft lean CLAY | 0 | | 2.2 | 8.5 | 4 | |
| | | | 2.2 | | | 15 | |
| | | 2.2 | | 0.7 | 8.6 | 26 | |
| | | | 2.9 | | 8.6 | 26 | |
| 2 | Loose silty sand | 2.9 | | | 9.0 | | 20 |
| | | | 3.5 | 0.6 | 9.0 | | 20 |
| 3 | Soft to slightly firm silty clay | 3.5 | | | 8.6 | 18 | |
| | | | 7.3 | 3.8 | 8.6 | 24 | |
| 4 | Medium dense silty clay | 7.3 | | 0.6 | 9.0 | | 20 |
| | | | 7.9 | | 9.0 | | 20 |
| 5 | Slightly firm silty clay | 7.9 | | 1.3 | 8.6 | 30 | |
| | | | 9.2 | | 8.6 | 30 | |
| 6 | Dense silty sand | 9.2 | | 2 | 9.4 | | 25 |
| | | | 12.2 | | 9.4 | | 25 |
| 7 | Slightly to moderately stiff | 12.2 | | 2.3 | 9.6 | 40 | |
| | silty clay | | 14.5 | | 9.6 | 50 | |
| 8 | Dense sandy silty clay and | 14.5 | | 5.4 | 9.8 | | 25 |
| | silty clay | | 19.9 | | 9.8 | | 25 |
| 9 | Stiff to very stiff silty clay | 19.9 | | 1.3 | 9.6 | 50 | |
| | | | 21.2 | | 9.6 | 50 | |
| 10 | Dense silty sand | 21.2 | | 7.6 | 9.8 | | 25 |
| | | | 28.8 | | 9.8 | | 25 |
| 11 | Stiff to very stiff silty clay | 28.8 | | 3.2 | 9.7 | 110 | |
| | | | 32 | | 9.7 | 110 | |
| 12 | Dense silty sand | 32 | | 8.7 | 9.8 | | 25 |
| | | | 40.7 | | 9.8 | | 25 |

This table includes several key parameters such as depth, soil geological description, submerged unit weight, and undrained shear strength. Each submerged unit weight data point is a consolidated analysis of all measured data within that soil layer, while each undrained shear strength integrates all experimental and CPT measurement into a single feature parameter. As an example, layer l's depth ranges from 0 to 2.9 meters. Two shear strength feature values for 0-2.2 meters and 2.2-2.9 meters are inferred from CPT, the collected manual vane test, electric vane test, UU triaxial compression test, CD multistage triaxial compression test, and CU multistage triaxial compression test data.

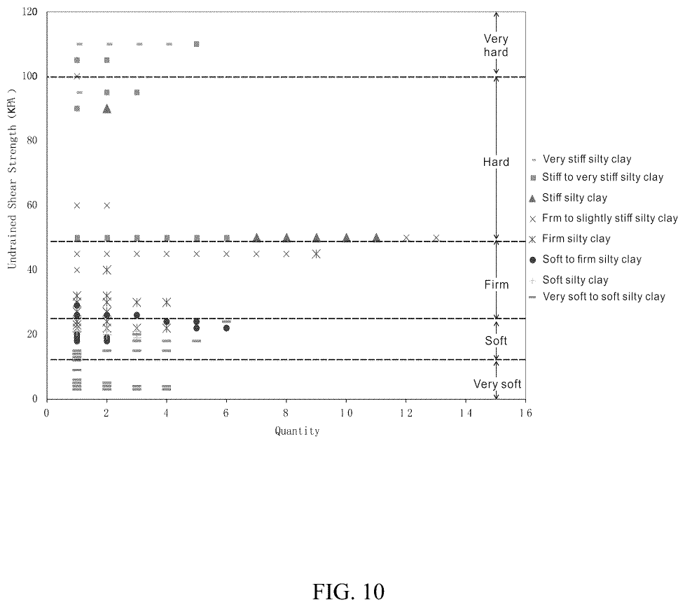

FIG. 10 illustrates that it is vague if classification is based solely on the single parameter of shear strength. Numerous data points are found on the boundary of classification. For instance, 13 data points lie on the boundary of firm and stiff classifications at 50 kPa. According to existing manual soil geological descriptions, these points vary from firm to very stiff clay (Table 1). Additionally, there are notable discrepancies between existing soil descriptions and shear strength-based classification; for example, 6 data points defined as “stiff to very stiff silty clay” fall between the firm and hard boundary, 7 data points identified as “firm silty clay” appear in the soft range, and 10 data points labeled as “very soft silty clay” are also categorized within the soft range. These inconsistencies demonstrate the limitations of using single-parameter shear strength classification in accurately describing soil properties and underscore the subjectivity of manual classification.

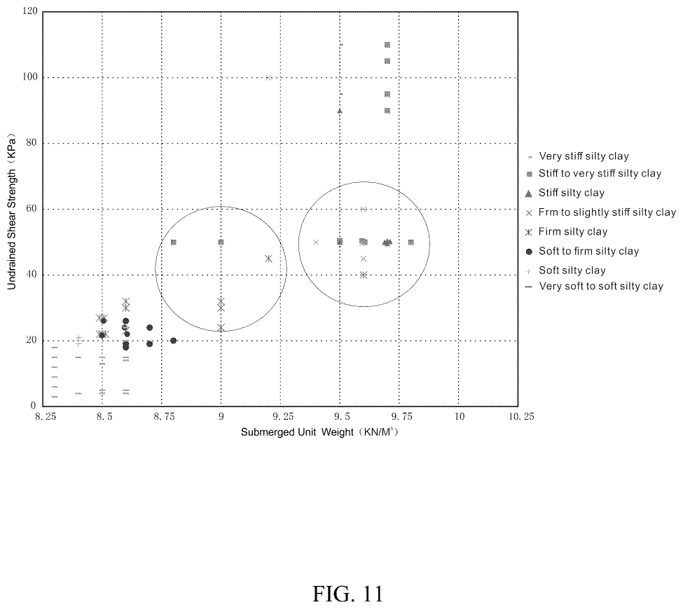

This invention employed dual-parameter cluster analysis, considering both mechanical and physical parameters. Initially, two key parameters, undrained shear strength and submerged unit weight, were chosen from the dataset. These parameters to a large extent reflect the mechanical and physical features of the soil and serve as inputs for the clustering process. In contrast to FIG. 10, FIG. 11 demonstrates that the 13 data points at 50 kPa exhibit different submerged unit weights.

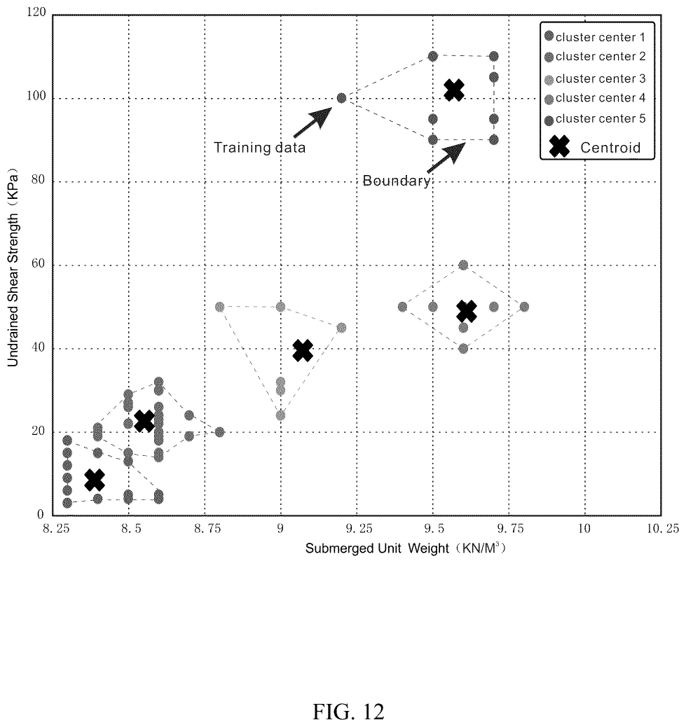

The next step is Step S20, which is to build a dual-parameter cluster model using the training set and a clustering analysis algorithm, followed by performing cluster analysis on the training set. Among various clustering algorithms available, the K-means method was selected as an example. Prior to running the K-means algorithm, it is necessary to determine the appropriate number of clusters. This decision can be aided by some commonly used artificial intelligence methods, such as the elbow method and silhouette coefficients, integrated with geological analysis. Through 2-3 iterations, this process ensures the selection of a cluster number that is most appropriate both geologically and from an engineering perspective (FIG. 12).

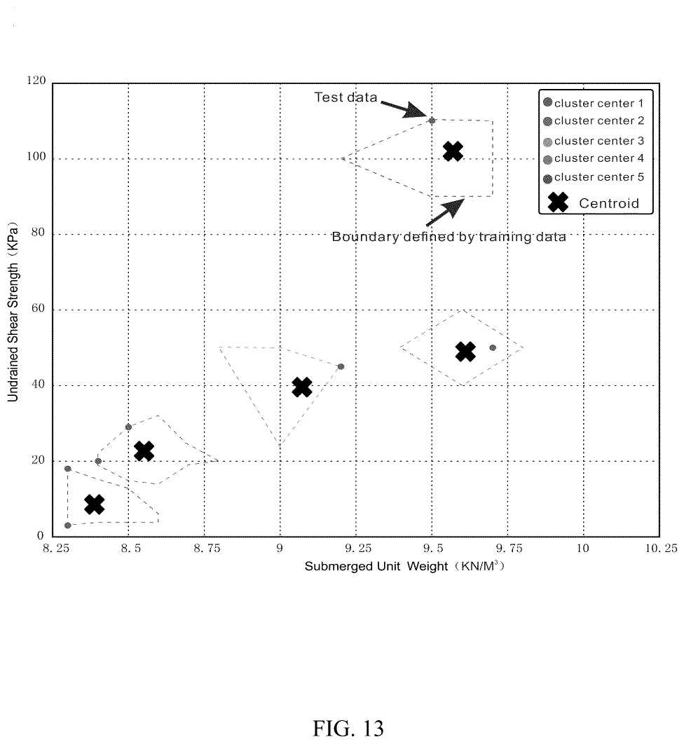

After conducting the dual-parameter cluster analysis, the next step, Step S30, requires the establishment of appropriate classification standards based on the clustering outcomes. Cluster analysis was performed on the testing dataset. FIG. 13 shows all test data fall within the boundaries of five categories.

Since multiple cluster centers were identified, the shear strength values at these centers did not match the traditional categories of very soft, soft, firm, stiff, and very stiff exactly but were rather near the boundaries of these categories. For example, the clustering results are shown in FIG. 12 and Table 2. It was observed that cluster center 2 had a shear strength of 22.53, cluster center 4 at 48.89, and cluster center 5 at 101.25, all of which are near the boundaries of traditional classification ranges.

TABLE 2
Soil classification, description, and designed
parameters at each cluster center

| Cluster | | Submerged | Undrained |
|---|---|---|---|
| center | Soil description | unit weight | shear strength |
| 1 | Very Soft to soft silty clay | 8.40 | 8.29 |
| 2 | Soft to firm silty clay | 8.56 | 22.53 |
| 3 | firm silty clay | 9.06 | 39.00 |
| 4 | Firm to stiff silty clay | 9.61 | 48.89 |
| 5 | Stiff to very stiff silty clay | 9.58 | 101.25 |

To address the discrepancies observed, this invention integrates the feature parameter values of cluster centers with the numerical ranges of traditional classification standards to establish new classification criteria. As illustrated in FIG. 12, while traditional standards divide into five grades: very soft, soft, firm, stiff, and very stiff, the figure also indicates numerous data points straddling the boundaries between soft and firm, firm and stiff, and stiff and very stiff. Although the one-dimensional mechanical parameter perspective originally divided the soil classification into 8 categories, these did not align perfectly with the 4 categories determined by artificial intelligence techniques. Consequently, the classification standards were redefined to fully incorporate both the analysis results and standard specification numerical ranges, ultimately adopting 5 soil types that more accurately represent the actual geological conditions.

The final step, Step S40, involves applying the dual-parameter cluster model once the accuracy satisfies predefined criteria. Additional soil data are then classified using this model. Initially, 11 borehole datasets are utilized for training and validating the model. After training, the model is further applied to classify soil data from the remaining boreholes.

What is claimed is:

1. A geotechnical/geological classification method based on dual-parameter cluster analysis, comprising the following steps:
Acquiring geotechnical data, preprocessing the data, and extracting feature parameters to obtain two parameter data, and dividing the dual-parameter data into a training set and a testing set;
Constructing a dual-parameter clustering model based on the clustering analysis algorithm, and performing cluster analysis on the training data to obtain clustering results;
Formulating classification standards based on the clustering results, verifying the results using the testing set, and evaluating the classification standards based on the verified results;
When the result reaches the preset accuracy, inputting the acquired data into the dual-parameter clustering model to obtain geotechnical classification results.

2. The geotechnical classification method based on dual-parameter cluster analysis according to claim 1, characterized in that the acquisition of geotechnical data, preprocessing of the data, and extraction of feature parameters to obtain dual-parameter data, and dividing the dual-parameter data into a training set and a test set, specifically includes:
Collecting geotechnical samples and conducting mechanical parameter tests and physical parameter tests to obtain the geotechnical data;
Preprocessing the geotechnical data, wherein the preprocessing includes data cleaning, data transformation, and data normalization;
Extracting feature parameters from the preprocessed geotechnical data to obtain the dual-parameter data, and dividing the dual-parameter data into a training set and a test set, wherein the feature parameters include: geotechnical depth, soil description, submerged unit weight, and undrained shear strength.

3. The geotechnical classification method based on dual-parameter cluster analysis according to claim 2, characterized in that the preprocessing of the geotechnical data specifically includes:
Deleting missing values, outliers, or erroneous values in the geotechnical data according to preset rules to obtain the first set of geotechnical data;
Transforming the data in the first set of geotechnical data to unify them into the same measurement units, resulting in the second set of geotechnical data, wherein the second set of geotechnical data is the preprocessed geotechnical data.

4. The geotechnical classification method based on dual-parameter cluster analysis according to claim 1, characterized in that the construction of the dual-parameter clustering model based on the training set and the clustering analysis algorithm, and the clustering analysis of the training set according to the dual-parameter clustering model to obtain clustering results, specifically includes:
constructing the dual-parameter clustering model in combination with the clustering analysis algorithm, establishing a dual-parameter database, and inputting the training dataset into the model;
Determining the number of clusters according to the dual-parameter clustering model, and initializing the cluster centers based on the number of clusters to obtain a position for each initial cluster center;
Performing iterative clustering analysis on the data in the training dataset based on the distance between the data in the training dataset and the initial cluster centers in the two-dimensional feature space of the dual-parameter clustering model to obtain clustering results.

5. The geotechnical classification method based on dual-parameter cluster analysis according to claim 4, characterized in that the determination of the number of clusters based on the dual-parameter clustering model and the initialization of cluster centers based on the number of clusters to obtain positions of initial cluster centers in two-dimension space, specifically includes:
Determining the number of clusters according to the elbow method, silhouette coefficient, or other artificial intelligence methods, as well as the industry standards applied in the training dataset;
Randomly selecting data points from the training dataset as initial cluster centers based on the clustering analysis algorithm, and obtaining all positions of initial cluster centers in the two-dimension space according to the determined number of clusters.

6. The geotechnical classification method based on dual-parameter cluster analysis according to claim 4, characterized in that the iterative cluster analysis of the data in the training set based on the distance between the data in the training set and the initial cluster centers in the two-dimensional feature space of the dual-parameter clustering model to obtain clustering results specifically includes:
Calculating the distance between the data in the training dataset and the initial cluster centers in the two-dimensional feature space, and assigning the data to the nearest initial cluster center to obtain initial clustering results;
Calculating the average value of all data points in the initial clustering results to obtain the centroid, and using the centroid as the new cluster center;
Iteratively calculating the distance between all data points of the initial clustering results and the new cluster centers, and updating the new cluster centers based on the distance until the new cluster centers no longer changes or the iterations reach a preset number, thereby obtaining the clustering results.

7. The geotechnical classification method based on dual-parameter cluster analysis according to claim 1, characterized in that the formulation of classification standards based on the clustering results, the verification of the clustering results using the test dataset, the obtaining of verification results, and the judgment of the accuracy of the classification standards based on the verification results specifically includes:
Formulating classification standards based on the clustering results and the numerical range of standard specifications, and inputting the test set into the dual-parameter clustering model;
Performing cluster analysis on the test set using the dual-parameter clustering model to obtain results for verification;
Judging the accuracy of the classification standards based on the test results by determining whether all data in the test set are assigned to the classification standards, and if so, confirming that the accuracy of the classification standards meets the preset requirements.

8. A geotechnical classification system based on dual-parameter cluster analysis, characterized in that the geotechnical classification system based on dual-parameter cluster analysis includes:
A data acquisition and preprocessing module, used for acquiring geotechnical data, preprocessing the data, extracting feature parameters to obtain dual-parameter data, and dividing the dual-parameter data into a training set and a test set;
A clustering model construction module, used for constructing a dual-parameter clustering model based on the training set and clustering analysis algorithm, and performing cluster analysis on the training set according to the dual-parameter clustering model to obtain clustering results;
A classification standards verification module, used for formulating classification standards based on the clustering results, verifying the clustering results using the test set to obtain verification results, and judging the accuracy of the classification standards based on the verification results;
A classification application module, used for acquiring more geotechnical data and inputting the geotechnical data into the dual-parameter clustering model to obtain geotechnical classification results when the accuracy meets the preset requirements.
